(19) United States
(12) Patent Application Publication
(10) Pub. No.: US 2010/0158813 A1
(43) Pub. Date: Jun. 24, 2010

(54) MICROBUBBLES AS DRUG DELIVERY DEVICE

(76) Inventors: Gaio Paradossi, Rome (IT); Francesca Cavalieri, Rome (IT)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 12/376,342

(22) PCT Filed: Aug. 3, 2007
(86) PCT No.: PCT/EP2007/006886

§ 371 Date: Mar. 1, 2010

(30) Foreign Application Priority Data
Aug. 4, 2006 (IT) BO2006A000593

(51) Int. Cl. A61K 33/00; A61K 33/04; A61K 49/12; A61P 7/02; C08F 8/28

(52) U.S. Cl. 424/9.52; 424/718; 424/699; 424/708; 424/600; 525/56

(57) ABSTRACT

A method of making PVA microbubbles including a functionalisation step in which PVA polymeric chains are functionalised at their ends with aldehyde groups, and a subsequent cross-linking step, in which in an air-aqueous solution emulsion with a pH between 4.5 and 5.5 the previously functionalised PVA polymeric chains cross-link by means of an acetalisation reaction, thereby forming the microbubbles. The microbubbles produced are subsequently subjected to a lyophilising step, a filling step in which medicinal gas is introduced and a restoration step of the microbubbles by adding an aqueous solution.

The present invention concerns the production of microbubbles and their filling with medicinal gases.

The role that gases play in medicine, either for diagnostic or treatment purposes, is continuously increasing. It is, however, sometimes difficult to deliver the gases to a patient in an appropriate form and an optimal dosage. Therefore, it is necessary to make drug carriers available that provide patients with medicinal gases under optimal conditions.

In this regard it has been described in EP 0 921 807 B1 that gas mixtures containing hydrogen gas may be administered in liposomes, microparticles or microcapsules to patients. The administration of liquids in microcapsules or microspheres is already known since several years (U.S. Pat. No. 6,911,219 B2, EP 1 263 801 B1, EP 1 263 802 B1, EP 0 332 175 A2).

The potential displayed by microbubbles with determined dimensional characteristics has been known for a long time, especially in the medical field, from both a diagnostic and therapeutic point of view. In fact, microbubbles have the advantages of exhibiting a high interface surface, of being stable and of being easily separatable from the reaction environment.

From the therapeutic point of view, microbubbles may be potential drug carriers inside the human body. In fact, microbubbles may be administered as injectable systems or taken orally as capsules or hydrogel. In this regard, the microbubbles are capable of incorporating the drug and upon reaching the tissue of interest releasing it when exposed to ultrasound.

The inventors found that polyvinyl alcohol (PVA) microbubbles offer a series of advantages, especially in terms of ease of manufacture, stability and the possibility of being superficially functionalised.

Recently, the formulation and the characteristics of air-filled and polymer shelled microballoons originating from a crosslinking reaction of poly (vinyl alcohol) at the air/water interface has been described (Cavalieri et al., Langmuir 21, 8758-8764 (2005)) By varying the synthesis conditions, like temperature and pH of the medium, some modulations of the size and shell thickness are possible, but typically the resulting bubbles have an average size of 4±1 μm and a shell thickness of about 0.6 μm, i.e. almost all particles have a size smaller than a red blood cell. Previous biocompatibility and cytotoxicity tests carried out on cancer cell lines has shown that the presence of PVA microbubbles did not affect the growth and morphology of cells, suggesting a favorable interaction of these microparticles with living cells (Cavalieri et al., Biomacromolecules 7, 604-611 (2006)).

These PVA microbubbles are made by functionalizing PVA chain ends with aldehyde groups and by subsequently causing acetalisation cross-linking in an emulsion made of air and aqueous solution (G. Paradossi et al., Biomacromolecules 2002, 3, 1255; F, Cavalieri, G. Paradossi, et al. Langmuir 2005, 21, 875B). As known in organic chemistry, acetalisation reactions are carried out under acid catalysis which then may be interrupted by neutralisation. When irradiated with ultrasound, these PVA microbubbles present a shell breaking threshold whose value is close to that of plasma membrane breaking. The shell breaking threshold is intended as the minimum pressure value at which the polymeric wall undergoes breaking under the action of the ultrasound. This characteristic involves serious problems for the use of these microbubbles as drug carriers since the ultrasonic cavitation action needed for the release of the drug could require such values which may also damage the surrounding cells.

Therefore, there was a need to make available PVA microbubbles which had a shell breaking threshold significantly lower than that of cells, in order to be able to use the microbubbles themselves as drug carriers.

Thus, the object of the present invention is to provide a method of making PVA microbubbles which do not damage surrounding cells if they are delivered as gas containing drug carriers to a patient.

Thus, the present invention concerns a method of making PVA microbubbles including:
a functionalisation step in which PVA polymeric chains are functionalised at their ends with aldehyde groups, and
a subsequent cross-linking step in which in an air-aqueous solution emulsion the previously functionalised PVA polymeric chains are cross-linked by means of an acetalisation reaction, thus forming said microbubbles, said method being characterised in that in said cross-linking step the aqueous solution has a pH of between 4.5 and 5.5.

A further object of the present invention is a method of filling the PVA microbubbles with medicinal gases, characterised in that it includes a step of lyophilising said microbubbles, a subsequent filling step in which the gas is introduced inside said microbubbles and into their shells, and a subsequent step of restoring said microbubbles by adding an aqueous solution.

Another object of the present invention is to provide PVA microbubbles filled with medicinal gases. These microbubbles are particularly interesting to be capable of releasing the gases locally and at effective concentrations.

As used herein, the term “microbubbles” is meant to indicate polymer based hollow colloidal microparticles capable of holding gas internally.

Polyvinylalcohol is a polymer prepared from polyvinyl acetates by the replacement of the acetate groups with hydroxyl groups, preferably having a 70 to 100 mole % hydrolysis rate. Also, two or more polyvinyl alcohols with different hydrolysis ratios may be us as a mixture. Polyvinylalcohols can be obtained from commercial chemical suppliers such as Aldrich, Fluka or Sigma. According to a preferred embodiment, the PVA polymeric chains have an average molecular weight of between 30000 and 200000, more preferably between 30000 and 100000 and most preferably between 30000 and 80000.

In the functionalization step a 1-10%, preferably 1.5-5%, most preferably a 2% (w/w) aqueous solution of PVA is prepared and an oxidizing agent (e.g. NalO₄) at a final concentration of 0.05-1% (w/w), most preferably 0.2% (w/w), is added. This solution is kept at an elevated temperature of 50-90° C., preferably about 80° C. for at least 30 minutes, preferably 1 hour. In this manner the PVA polymeric chain ends are functionalized with aldehyde groups.

To the aqueous solution containing the functionalized PVA an aqueous acid, i.e. diluted sulphuric acid, phosphoric acid, hydrochloric acid or nitric acid, is added to obtain a pH of about 4.5 to 5.5., or preferably, using the limited acidity of distilled water, between 4.5 and 5.5 (most preferably about 5.0).

The aqueous solution containing the functionalized PVA and having a pH between 4.5 and 5.5 is then submitted to strong stirring at 5000-15000 rpm, preferably 7000-10000 rpm, most preferably at about 8000 rpm. The stirring may be carried out e.g. by means of an “Ultra Turrax” homogenizer for a period of 1-4 hours, preferably 2-3 hours, most preferably about 2 hours. This step is preferably carried out at room temperature although temperatures between 5-30° C. may be also applied. Then, the floating particles are separated from the precipitated material and washed, obtaining an aqueous suspension comprising 10⁶-10⁷ microbubblesper ml. The thus obtained microbubbles are comprised of a polymeric membrane which holds air and whose thickness is between 0.5 and 0.7 μm, and show an average diameter of between 3.5 and 5.5 μm.

According to a standard method the shell breaking threshold is studied as well as the mechanical index of the above-mentioned microbubbles by applying ultrasounds at a frequency of 2.2 MHz. The mechanical index (MI) is directly proportional to pressure and inversely proportional to the square root of the frequency of the ultrasounds and must be generally lower than 1.9 in medical diagnostics.

The shell breaking threshold measured on the prepared microbubbles is below 1.00 MPa, preferably between 0.90 and 0.98 MPa, most preferably about 0.95 MPa, corresponding to an MI of 0.50 to 0.60, preferably about 0.53 (Pecorari C., Cavalieri F., Paradossi G., Brismar T. Proceedings of the 2007 International Congress on Ultrasonics, Wien, 2007).

As it emerges from the above-described MI values, the microbubbles made with the method of the present invention, unlike those made with the method of the prior art, allow for releasing any drug with which they are loaded without any damage to the cells.

A further advantage of the method of the present invention lies in its ease of manufacture, especially taking into consideration that the preferred pH is that of water and that the best results are obtained by operating at room temperature.

For loading of the microbubbles with a medicinal gas an aqueous suspension of PVA microbubbles is frozen, e.g. in liquid nitrogen, and lyophilised. Thus, porous microparticles are obtained. The lyophilised microparticles are introduced into a reaction vessel, e.g. a steel reactor, subjected to a flux of noble gas, e.g. an argon flux, with the aim of creating an inert environment and subsequently loaded with a medicinal gas at the pressure of 1.0-2.0 atm , preferably 1.5 atm, for 1-4 hours, preferably 2-3 hours. At the end of the process, the medicinal gas is evacuated from the reactor by means of an noble gas flux, e.g. argon flux, and the microparticles loaded with the medicinal gas are stored in an inert environment.

The presence of the medicinal gas in the microbubbles may be detected by means of electron spin resonance (ESR) spectroscopy (preferably at room temperature) and directly on PVA microbubbles lyophilisates and colorimetric analysis (Griess assay) of aqueous suspensions.

As may be obvious to one skilled in the art, the method of loading PVA microbubbles with medicinal gas is independent from the type of process with which the microbubbles themselves are made.

The inventors have developed a new concept of drug delivery in which medicinal gas release can be performed by means of polymer shelled microbubbles. Responsiveness of this drug platform to ultrasound can be suitably exploited for enhancing the gas release from the delivery device by bursting of the microparticles upon sonification.

Ultrasound contrast agents are examples of micro/nano imaging devices that already are in medical use. Ultrasound contrast agents are made of a lipidic or proteinaceous shell with a core containing a stabilizing gas. They consist of millions of micron sized bubbles that are injected to the bloodstream. If the bubbles, providing the contrast effect in the ultrasound imaging methodologies, can be loaded with drugs local release and local non-invasive therapy will be possible. To improve their diagnostic and therapeutic features they should also be able to target the tissue by chemical binding or affinity. Several issues must be addressed in developing these devices such as longer shelf and circulation life, chemical versatility of the surface for easy modifications and a large payload capacity. Moreover, ultrasound scattering efficiency for high quality imaging must be optimized and the occurrence of inertial cavitation must be kept at a mechanical index value (MI) below 1.0 to accomplish drug release by ultrasound irradiation without tissue damage.

Decoration of the external surface of these bubbles with several molecules is also possible. This opens a clue on the coupling reactions that can be used for the attachment of ligands to the surface of microbubbles. For example it is possible to have an adhesion promoter (e.g. CM dextran, collagen, DEAE dextran, gelatin, glucosaminoglycans, chitosan, polypetides and proteins, fibronectins, lectins, etc.) and/or a marking agent (e.g. dyes and fluorescent labeling agents, imaging agents, contrasting agents) and/or targeting ligands (e.g. antibodies or folate galactose) bound to the surface.

The concept of the present invention in view of the obtainment of a micro device for the in situ delivery of medicinal gases is (i) to inject in the blood stream microbubbles with suitable targeting for clots, (ii) to monitor the clot by ultrasound contrast enhancing, (iii) to insonify the microbubbles up to the rupture threshold by increasing the ultrasound amplitude, (iv) to deliver the gas in the clot domain in order to disrupt it or to facilitate its disruption.

The inventors show in the present application new structural features and the successful loading of PVA based microbubbles with medicinal gases proving that such device can be considered a truly multifunctional agent for both diagnostic and therapeutic purposes.

One of the gases becoming more and more important in medicine is nitrous oxide (NO). NO plays a role in controlling arterial thrombosis and in cardiovascular diseases by the inhibition of the platelet aggregation process. This molecule acts as deactivating signal of the protein membrane integrins, the major platelet adhesion receptors. The localized production of NO, naturally occurring in arterial vessels, is carried out by the NO synthase enzymatic system. The inhibition of the platelet aggregation in the coagulation cascade process is due to the antagonistic action of NO towards integrin-fibrinogen induced platelets adhesion. Furthermore, NO containing gaseous mixtures are known for the treatment of reversible pulmonic vasoconstriction and bronchoconstriction (WO-A-92/10228). A further medical indication for the administration of NO is the treatment of perinatal aspiration syndrome.

Other gases that are useful for medicinal purposes and may be filled into the microbubbles are CO, hydrogen, oxygen, helium, xenon, H₂S, N₂O, argon, and any mixtures thereof. In this regard mixtures of NO and H; NO and xenon; NO, xenon, He/oxygen (i.e. heliox) and CO are particularly preferred.

It is known that carbon monoxide (CO) has an important role as signal transducer in certain physiological processes, in particular in the cardiovascular system. Furthermore, it helps to avoid graft-versus-host reactions after organ transplantation and diminishes damages of ischemia.

Hydrogen containing gas mixtures are useful for the treatment of lung diseases and certain inflammatory diseases. Deuterium (heavy hydrogen) has been proven to have a toxic effect on tumor cells. Combinations of hydrogen and nitrous oxide gas may be used for the preparation of a medicament for treating reversible or irreversible pulmonic vasoconstriction, bronchoconstriction and inflammatory diseases of the lung and COPD (EP-A-0 921 807).

Oxygen and air are known to have a positive effect on all vital functions. Medicinal oxygen is useful for the treatment of all types of shortness of breath and oxygen deficiency. These problems may be caused by pneumonia, lung infarction, lung fibrosis, lung oedema, lung cancer/metastasis, heart infarction, Angina pectoris, emphysema, shock, decompression disease, anaemia, hypoxia, poisoning with CO and/or CN, Myasthenia gravis, etc.

Xenon and N₂O are each known as medicinal gases having an anaesthetic and/or analgesic effect. Furthermore, both gases have been suggested to have neuroprotective effects (David et al., J. of Cerebral Blood Flow Metabolism, 23, pp. 1168-1173 (2003)).

Also the gas Argon has been suggested to treat neurointoxications (US-2005/0152988 A1).

Helium, in particular a mixture of helium and oxygen (Heliox), has recently been found to reduce infarct volume in a rat model of focal ischemia (Pan et al., Experimental Neurology, in press, 2007).

H₂S is known to induce stasis in cells, tissues, and/or organs in vivo or in an organism overall so as to preserve and/or protect them. This can be useful in therapeutic methods for organ transplantation, hyperthermia, wound healing, hemorrhagic shock, cardioplegia for bypass surgery, neurodegeneration, hypothermia, and cancer is provided (WO-A-2005/041655).

Furthermore, the new method of making PVA microbubbles allows a targeted administration of low doses of gases to a patient. This will make it possible to find new medicinal uses for gases not yet considered as medicinal due to their toxic/damaging effects. For instance chlorine gas, acetylene, ethylene or any other gas could be administered in low doses to a target point in a patient without having any negative systemic effects.

The invention is further described with reference to the Figures, which show:

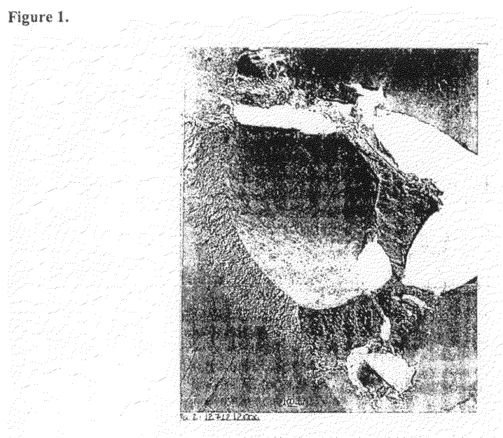

FIG. 1: Electron micrograph of freeze-fractured microbubble fabricated at pH 5 at room temperature showing a shell thickness of 0.4 □m with a microstructure consisting of PVA microfibrills.

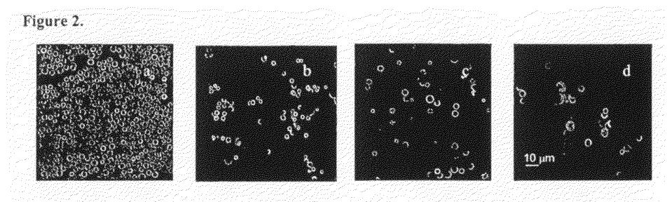

FIG. 2: NAPSS concentration: 0%, 3%, 7% and 13% (w/v); a, b, c, d, respectively. Scale in (d) is the same for all images.

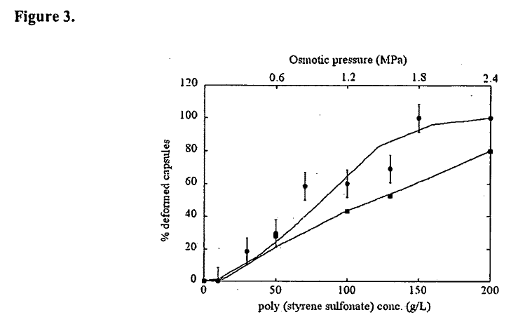

FIG. 3: Percent of deformed capsules by osmotic stress as a function of the concentration of polyelectrolyte. • MCpH2C; ▪ MCpH5C. Line is a guide for eye.

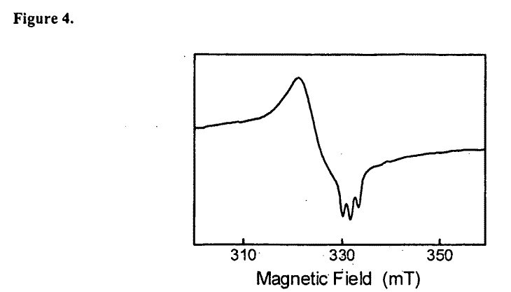

FIG. 4: EPR spectrum of myoglobin-nytrosyl complex at 100K in NO loaded microbubbles suspensions.

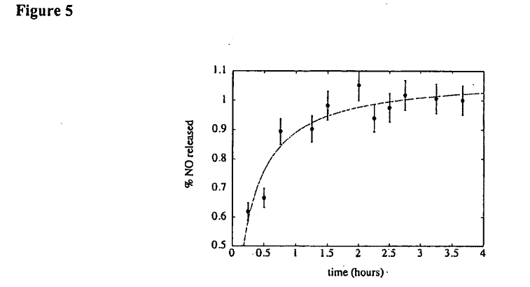

FIG. 5: Release of NO by microbubbles measured as nitrites by Griess essay.

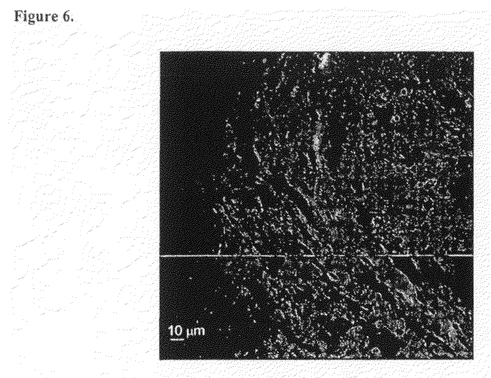

FIG. 6: CLSM image of a clot formed in vitro with RBITC tagged platelets (red dots) and entrapped unloaded microbubbles (red rings).

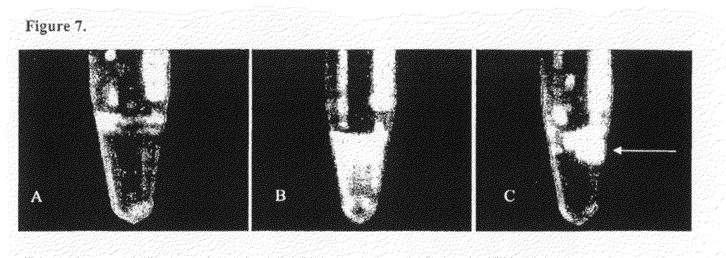

FIG. 7: (A) Clotting medium in the presence of NO loaded microbubbles used immediately after reaction container opening (time 0 condition); (B) Clotting medium in the presence of NO loaded microbubbles after 1 hour from the reaction container opening; (C) NO loaded microbubbles after 2 hours from the reaction container do not prevent the formation of a clot as indicated by the arrow. Pictures were taken one hour after microbubbles addition to the clotting medium.

### DETAILED DESCRIPTION OF THE PRESENT INVENTION ON THE BASIS OF PREFERRED EMBODIMENTS

PVA based microbubbles fabrication consists in a coupling reaction at the water-air interface of an aqueous solution of modified poly (vinyl alcohol) bearing two aldehydes as terminal groups. This process is an acetalization leading to the crosslinking between some of the backbone hydroxyls and the chain ends. When the reaction medium is stirred at high shear rate and due to the foaming properties of PVA, part of the crosslinked polymer chains goes into the formation of the microbubbles shells. At the end of the process, a stable colloidal suspension, floating at the meniscus of the aqueous reaction medium, formed by micron sized particles with an air filled core and with a polymer shell is obtained. Purification of this dispersion is easily accomplished by replacing the reaction medium with double distilled water.

Three samples have been investigated in the present application: microbubbles prepared at 5° C., at pH 2 and at pH 5, MBpH2C and MBpH5C, respectively, and at pH 5 at room temperature, MBpH5RT.

The morphological characterization of the microbubbles has been carried out by laser scanning confocal microscopy and freeze fracture electron microscopy. CLSM allows the observation of the equatorial plane of individual microbubbles providing an evaluation of the average size of their diameters and shells. Fluorescent labeling of microbubbles was obtained by FITC and RBITC coupling to the microparticles surface. Freeze-fracture electron microscopy allowed a precise evaluation of the microbubbles shell thickness .

The conversion of air-filled microparticles to solvent-filled microcapsules was carried out as a method to evaluate the elasticity of the particle polymer shell exposed to osmotic stress for the presence of sodium poly (styrene sulfonate), NaPSS, at known concentration in the external aqueous medium.

Loading of a medicinal gas was performed on a freeze-dried sample of microbubble aqueous suspension placed in a stainless steel reaction vessel and pressurized with the gas at 2 bar for 3 hours. Loading capacity and time release was measured by Griess assay. The gas was detected by dispersing the loaded microbubbles and recording the EPR spectrum in the presence of myoglobin, Mb. The gas-Mb complex spectrum is diagnostic for the presence of the gas and the EPR characterization is well known in the art.

A blank experiment was carried out by forming a clot in the presence of unloaded microbubbles. The same procedure was repeated with freshly prepared gas-loaded microbubbles and with gas-loaded microbubbles exposed to air for 1 and 2 hours.

The above mentioned steps are described in more detail below:

Microbubbles Characterization

The main structural requirement for using microbubbles as e.g. an ultrasound contrast agent are dictated by their injectability in the circulatory system. Therefore, their size should not overcome the capillary lumen, i.e. they should not be bigger than a red blood cell and they should have a limited and controlled polydispersity.

Structural characterization of the PVA shelled microbubbles has been based mainly on confocal microscopy observations (Cavalieri, F et al., Langmuir 21, 8758-8764 (2005)). An average diameter of 5±1 μm and a shell thickness of 0.7 μm were determined for microbubbles synthesized in the presence of sulphuric acid as a catalyst at 5° C. This approach is well suited for the determination of the particle average diameter, but it lacks the resolution needed for a reliable determination of the shell thickness. With this goal in mind, the inventors have carried out a freeze fracture electron microscopy analysis with a typical resolution of 2 nm on microbubbles obtained at pH 5 at room temperature.

Fractured microbubbles were analyzed to characterize the surface morphology of the particles and to evaluate the shell thickness (FIG. 1.) . It can be observed that the shell is a corona surrounding the particle spheroidal section with PVA fibrils radially protruding. The thickness of the shell is 0.4±0.1 μm. As evidenced in the circled area, an outer region characterized by loosely arranged PVA fibrils and an inner region where the polymer fibrils are organized in a more compact fashion can be distinguished. The colloidal stability of this system may be attributed to the presence of polymer chains extending into the solution and forming the “hairy” surface.

Microbubbles to Microcapsules Conversion

An interesting feature of PVA based microbubbles resides in the possibility to transform them into microcapsules by dispersing the bubbles in DMSO. PVA based microbubbles are stable in water for months. However, when they are dispersed in DMSO, the empty cavity is filled by the organic phase in few hours. In fact, DMSO is a good solvent for PVA and the shell of the microbubbles is expected to swell in this medium. The consequent increase in the pore size facilitates the permeation of DMSO in the inner cavity transforming the bubbles into capsules. At this point the encapsulated DMSO can be replaced with other solvents, i.e. water, by dispersing the particles in the new medium. This feature makes possible the use PVA-shelled capsules as carrier for water soluble drugs. Average diameter and shell thickness were obtained by statistical analysis of fluorescence profiles of confocal microscopy images on 100-200 microparticles in the form of (i) microcapsules and (ii) microbubbles using confocal microscopy images (see Table 1).

TABLE 1
Structural parameters of microbubbles (MB)
and microcapsules (MC) determined by CLSM.

| | External | Shell | | External | Shell |
|---|---|---|---|---|---|
| | diameter, | thickness, | | diameter, | thickness, |
| MB type | μm | μm | MC type | μm | μm |
| MBpH2C | 3.0 ± 0.8 | 0.7 ± 0.3 | MCpH2C | 4 ± 1 | 0.6 ± 0.3 |
| MBpH5RT | 4.6 ± 0.9 | 0.6 ± 0.3 | MCpH5RT | 4.6 ± 0.8 | 0.9 ± 0.3 |
| MBpH5C | 2.7 ± 0.5 | 0.5 ± 0.3 | MCpH5C | 3.3 ± 0.6 | 0.6 ± 0.3 |

Microbubbles prepared at 5° C., in the presence of acidic catalyst or in water, MBpH2C and MBpH5C, respectively, exhibit a smaller diameter compared to those synthesized at room temperature due to the higher gas solubility at lower temperatures. However, a slight increase of microbubbles overall size was observed when their gas containing core was permeated by solvent, converting them into microcapsules.

The conversion into capsules offers the opportunity to use osmotic stress for evaluating the shell elasticity of PVA based microbubbles. Shell flexibility of microbubbles was qualitatively observed also in densely packed aqueous dispersions, where an almost hexagonal arrangement is reached between the particles. In this crowded arrangement the shells of microbubbles display a flattening in correspondence of the contact points between the bubbles.

The experiment was carried out by equilibrating the water containing PVA microcapsules against aqueous solutions at increasing concentrations of the polyelectrolyte sodium poly (styrene sulfonate), NaPSS, with a molecular weight of 70,000 dalton in order to avoid any permeation of the polyelectrolyte through the microbubble shells.

The morphology of the microparticles at different external polyelectrolyte concentration was evaluated by CLSM as shown in FIG. 2 (a-d).

Once the osmotic pressure in the bulk is larger than in the internal cavity, the hydrostatic pressure difference tends to deform the microcapsules. Typical invagination can be noted in the micrographs of TRITC-labeled PVA shells exposed to the highest polyelectrolyte concentrations.

A statistical evaluation carried out on samples of 200 microcapsules yields (see FIG. 3) a threshold value of the osmotic pressure (corresponding to 50% of deformed capsules) at about 1.1 MPa, common to two microbubbles types, MBpH2C and MBpH5RT. MBpH5C type reaches the threshold value at a higher osmotic pressure.

Theoretical modeling relating this critical pressure to the mechanical properties of microcapsule shells was developed and applied for the study of microcapsules made by layer-by-layer polyelectrolytes adsorption (Gao, C. et al., Langmuir 17, 3491-3495 (2001)). In this approach capsules loose their Euler stability with consequent deformation when the work performed by external pressure is equal to the deformation energy (Gao, C. et al., Eur. Phys. J. E5, 21-27 (2001)). For this system the Young modulus, E, is:

$$E = \frac{3}{4} {{π}_{c} ( R / δ )}^{2} \qquad [ 1 ]$$

where π_{c} is the critical pressure at the buckling transition, i.e. when half of the sampled microbubbles are deformed, R is the microbubble radius and δ is the shell thickness determined by CLSM.

As shown in FIG. 3 about 10% MCpH5RT bubbles were deformed in the control sample, i.e. in the absence of any osmotic stress. In this case this plastic deformation effect was not included for the determination of the pressure threshold corresponding to the 50% of buckled capsules. Determination of Young modulus according to eq. 1 for the examined microcapsules is reported in Table 2.

TABLE 2
Critical osmotic pressure and Young moduli of microcapsules (MC).

| | Critical | Young |
|---|---|---|
| | Pressure | Modulus |
| MC type | (MPa) | (MPa) |
| MCpH2C | 1.1 | 9.5 |
| MCpH5RT | 0.9 | 4.5 |
| MCpH5C | 1.8 | 10 |

The elastic moduli obtained by osmotically induced buckling of microcapsules are in good agreement with the values reported for elastomeric films (Polymer Handbook, Brandrup et la., Eds. 1999)). However, they are much smaller than the values found with the same method for capsules prepared by layer-by-layer polyelectrolytes deposition.

In view of the potential use of these microbubbles as ultrasound diagnostic tool and as ultrasound responsive devices for localized drug release, these data allow a qualitative evaluation of the mechanical index, MI, at which microbubbles should break upon insonification. The operative definition of this parameter is (Apfel, R.E. et al., Ultrasound in Med. & Biol. 17, 179-185 (1991)):

$$MI = \frac{P}{\sqrt{F}} \qquad [ 2 ]$$

where F is the rarefactional pressure in MPa and F is the frequency in MHz of the ultrasound wave, respectively.

Microbubbles mechanical properties and responsiveness to ultrasounds are affected by the crosslinks density and average porosity of the polymer shells. Insight on porosity features of PVA shells synthesized in different conditions can be provided by size exclusion measurements of nearly monodispersed fluorescently labeled dextran fractions on PVA based capsules. As shown in Table 3, the average porosity of the capsules is larger for the shells prepared at pH 5 compared to those prepared at pH 2 (in aqueous H₂SO₄). A smaller pore size indicates a higher crosslinks density and suggests a higher surface stiffness.

TABLE 3
Determination of the porosity of PVA based microcapsules
by size exclusion measurements.

| Dextrans | | Penetration | Penetration | Penetration |
|---|---|---|---|---|
| molecular | | through | through | through |
| weights | Hydrodynamic | MCpH5C | MCpH5RT | MCpH2C |
| (g/mol) | radius (nm) | shell | shell | shell |
| 70,000 | 6.5 | no | No | No |
| 20,000 | 3.5 | no | No | No |
| 10,000 | 2.7 | — | Yes | No |
| 4,000 | 1.7 | yes | Yes | No |

This is in agreement with the higher modulus measured by osmotic stress on MBpH2C and on MCpH5C compared to the MCpH5RT (see Table 2). This finding indicates that sulfuric acid, used as catalyst, promotes a more efficient chemical crosslinking.

Microbubbles as Medicinal Gas Delivery Platform

In the following NO gas has been choosen as an exemplary gas. However, the obtained results and below statements also apply to any other medicinal gas or gas mixture.

NO loading of microbubbles was carried out in a stainless steel container by pressurizing freeze-dried bubbles with NO at 2 bars for 3 hours. The presence of NO adsorbed on the microbubbles shell was then monitored by adding freshly NO loaded microbubbles in an aqueous myoglobin solution in the presence of sodium dithionite to maintain reducing conditions. The EPR spectrum at 100 K (see FIG. 4) was indicative of the six coordinate NO-heme complex with the characteristic g₁=2.08, g₂=2.01 and g₃=1.98 values (Archer S., FASEB J. 7, 349-360 (1993)).

The NO release was evaluated indirectly by analyzing the nitrites derived from NO oxidation in aqueous medium by Griess colorimetric assay. The initial time lag in FIG. 5 refers to the time lapse occurring from the opening of the container to the transfer of the NO loaded microbubbles into PBS solution. An initial release burst of 60% is due to the oxidation of NO during this initial time lag. The remaining 40% of the total NO loaded in the micro bubbles is released in PBS in about 2 hours, a suitable time window for routine echographic manipulations. The average NO content per mg of microbubbles is 3.6 μmol.

Clotting in the Presence of NO Loaded Microbubbles.

NO has received increased attention in recent years by virtue of its biological functionalities. Presently this molecule is recognized as an important agent regulating vasodilation, neurotransmission and endothelium repairing.

To validate the concept of microbubbles as NO delivery platform the inventors have carried out in vitro tests by visualizing clot formation by CLSM in the presence of unloaded and NO loaded microbubbles. FIG. 6 shows the results of the blank experiment where the clot is visualized by tagging fibrinogen with FITC. Tagged platelets and entrapped microbubbles were labeled with RBITC, showed in FIG. 6 as red dots and red rings, respectively. This experiment indicates that the presence of the unloaded microbubbles does not inhibit clot formation.

Normally, the clot should develop macroscopically in ten minutes. In FIG. 7 (A), the clotting medium, freshly prepared and not showing any opalescence due to initial aggregation, was added with NO loaded microbubbles right after the opening of the container: the addition of NO loaded microbubbles substantially slowed down the clot formation. After 1 hour from the opening, the NO loaded microbubbles had lost some of the ability to prevent the clotting process (FIG. 7B). The formation of a stable fibrin gel-like network is therefore achieved. NO loaded microbubbles left in the atmosphere for two hours are not able to prevent the formation of clot as indicated by the arrow pointing a clot lump (FIG. 7C). All the pictures were taken 1 hour after the addition of the microbubbles to the clotting medium.

This is the first example of an in vitro NO localized delivery device and consequent activation of inhibitory signal for the regulation of platelets adhesiveness. The microdevice described here could supply the basis for the development of a multifunctional NO and other therapeutic gasses carrier and the possibility to deliver non gaseous drug molecules will be also considered. Finally, this work is meant to be a contribution to the arsenal of new tools for an implemented therapeutic approach where localization of the treatment conjugated to limited invasiveness is coupled to high efficiency in ultrasound imaging in the context of a spread out diagnostic approach as echography.

The invention is further described with regard to the following examples which serve an illustrative but not a limiting purpose, for a better understanding of the invention.

### EXAMPLE 1

### Microbubbles Fabrication

Materials. Poly (vinyl alcohol) (PVA) was a Sigma product (Germany) with number average molecular weight (Mn) of 35,000. Sodium poly (styrenesulfonate, sodium salt) (PSS) Mw 70000, Rhodamine B isothiocyanate, RBITC and Flurescein isothiocyanate isomer I (FITC) were Fluka products (Germany). Fluorescein isothiocynate labeled dextrans (FITC dextrans) with number average molecular weights of 4000, 10000, 20000, 70000 and labeling density of 0.004 mol of FITC/mol of glucose were also supplied by Sigma. Dimethyl sulfoxide (DMSO), sodium periodate and inorganic acids used for microbubbles preparation were RPE products from Carlo Erba (Italy).

Double distilled water with resistivity of 12.8 M Ohm·cm (MilliQ water) was used throughout this study.

The fabrication of PVA based microbubbles has been reported by Cavalieri, F. et al., Macromol. Symp. 234, 94-101 (2006). In summary, 2 g of PVA were dissolved in 100 ml of

MilliQ water and oxidized by sodium periodate. During high shear stirring ( [symbol] ) with an Ultra Turrax (IKA, Germany) the medium was maintained at pH 2 by H₂SO₄ in a iced water bath, MBpH2C, or at pH 5 at room temperature or in a iced water bath, MBpH5RT, MBpH5C, respectively.

### EXAMPLE 2

### Confocal Laser Scanning Microscopy (CLSM)

FITC and RBITC were used for fluorescent labeling of microbubbles, microcapsules and fibrinogen. Fluorescent dyes were added into the microbubbles suspension at a typical concentration of 10 μM, the mixture was stirred for 2 hours. Floating particles were washed by re-suspending them in MilliQ water several times. Fibrinogen was labeled with FITC in 0.1 carbonate buffer at pH 8.5 and FITC/protein weight ratio of 1.20. Confocal images were collected by a confocal laser scanning microscope, Nikon PCM 2000 (Nikon Instruments): a compact laser scanning microscope based on a galvanometer point-scanning mechanism, a single pinhole optical path and a multi-excitation module equipped with Spectra Physics Ar-ion laser (488 nm) and He-Ne laser (543.5 nm) sources. A 60x/1.4 oil immersion objective was used for the observations.

### EXAMPLE 3

### Freeze-Fracture Electron Microscopy

The analysis was carried out by Nano Analytical Laboratory, San Francisco (USA), on a microbubble sample prepared at room temperature at pH 5 in H₂O. The sample was quenched using sandwich technique and liquid nitrogen-cooled propane. Using this technique a cooling rate of 10,000 Kelvin/s is reached avoiding ice crystal formation and artifacts possibly caused by the cryofixation process. The cryo-fixed sample was stored in liquid nitrogen for less than 2 hours before processing. The fracturing process was carried out in a JEOL JED-9000 freeze-etching equipment (JEOL, Japan) and the exposed fracture planes were shadowed with Pt for 30 s in a angle of 25-35 degree and with carbon for 35 s (2 kV/ 60-70 mA, 10⁻⁵ Torr). The replicas produced in this way were cleaned with concentrated, fuming HNO₃ for 24 hours followed by repeating agitation with fresh chloroform/methanol (1:1 by vol.) at least 5 times. The replicas were then examined at a JEOL 100 CX or Philips CM 10 electron microscope.

### EXAMPLE 4

### EPR of NO Loaded Microbubbles and NO Release

Freshly prepared microbubbles were repeatedly rinsed with MilliQ quality water in order to dilute the non-reacted PVA. Microbubbles water suspension was freeze-dried and the resulting powder was placed in a stainless steel reactor. The vessel, connected to an NO tank by stainless steels luer-lock connections, was pressurized to 2 bar and left in this condition for 3 hours. Freeze-dried NO loaded microbubbles were suspended in a 1 mM myoglobin and sodium dithionite to assure reducing conditions. X band EPR spectra were recorded on a EMX Bruker spectrometer operating at T=100 K at 0.5 mT field modulation.

5 mg of NO loaded microbubbles were suspended in 7 ml of PBS, an aliquot of 200 □l was tested to quantify NO release by using Griess assay. Loading capacity was determined by means of a nitrite calibration curve in PBS. Calibration curves in PBS were carried out with NaNO₂ standard solution.

### EXAMPLE 5

### In Vitro Clots Formation

Materials for clotting:

Platelets were purchased from Helena Bioscience Europe, epinephrine, CaCl₂, glutathione, sodium dithionite, fibrinogen and thrombin were Sigma products used without further purification.

In a typical in vitro clot preparation, Tris buffered saline solution at pH 7.6 was used as solvent and for platelet reconstitution: 60 ml of lyophilized formalin-fixed platelets were reconstituted by adding 5 ml of the above mentioned medium, equilibrating for 10 min.

### EXAMPLE 6

### In Vitro Clot Tests

0.5 mg of freshly NO loaded microbubbles were suspended in a clotting solution composed by 60 μl of platelet suspension, 206 μl of 0.15 mM epinephrine, 30 μl of 17mM CaCl₂, 18 μl of 0.3 mM glutathione, 35 μl of 50 mg/ml fibrinogen, and 35 μl of 10 u/ml thrombin. The same test was carried out with NO loaded microbubbles after 1 and 2 hours of exposure to atmosphere. Blank experiment was carried out with unloaded microbubbles. Laser scanning confocal microscopy was used to distinguish between the clot mesostructure and the presence of the microbubbles by FITC tagging of fibrinogen, whereas RBITC was used for tagging platelets and microbubbles.

### EXAMPLE 7

### Microbubbles—Microcapsules Conversion

Microbubbles were converted into solvent filled microcapsule according to the procedure reported in the literature (Cavalieri, F. et al., Langmuir 21, 8758-8764 (2005)). Shortly, an aqueous suspension of microbubbles was exchanged with DMSO. After two days the particles sunk at the bottom of the test tube, indicating that the particles core was filled by the organic phase and that the conversion was completed. DMSO was then replaced with water by repeated washings. Microcapsules permeability to FITC-dextrans at different molecular weights (4000-70000) was followed by CLSM. Microcapsules were suspended in aqueous solution of dextran-FITC at concentration of 1 mg/ml overnight in order to assure macromolecule permeation into the capsule cavity.

Determination of capsules rupture by osmotic pressure stress was carried out by CLSM observation of capsules aqueous dispersion in the presence of sodium poly (styrene sulfonate), NaPSS, with Mw=70,000 at different concentration (1-20%). The osmotic pressure of NaPSS solutions was measured by means of membrane osmometer and calibration curve was used to evaluate the osmotic pressure during the buckling of as microcapsules. At least 200 microcapsules were counted and the deformation ratio was defined as the ratio of deformed capsules to total number of capsules. The critical PSS concentration was defined as the concentration required to induce a cup-like shape to 50% of intact microcapsules (Cavalieri et al., Langmuir 21, 8758-8764 (2005)).

1. A method of making polyvinyl alcohol (PVA) microbubbles including a functionalisation step in which PVA polymeric chains are functionalised at their ends with aldehyde groups, and a subsequent cross-linking step in which in an air-aqueous solution emulsion the previously functionalised PVA polymeric chains are cross-linked by means of an acetalisation reaction, thus forming said microbubbles; wherein in said cross-linking step the aqueous solution has a pH of between 4.5 and 5.5.

2. The method according to claim 1, wherein the PVA polymeric chains have an average molecular weight of between 30000 and 200,000.

3. The method according to claim 1 wherein said cross-linking step is carried out at room temperature.

4. PVA microbubbles obtainable according to the method of claim 1.

5. Microbubbles according to claim 4, wherein the PVA polymeric wall has a thickness of between 0.5 and 0.9 μm.

6. A method of using microbubbles of claim 4 as drug carriers comprising the steps of loading a drug in or onto the microbubbles and administering the microbubbles to a patient, wherein the drug is released without damage to the surrounding cells.

7. A method of filling the PVA microbubbles of claim 4 with at least one medicinal gas, comprising lyophilising said microbubbles, subsequently introducing a medicinal gas inside said microbubbles, and subsequently restoring said microbubbles by adding an aqueous solution.

8. The method according to claim 7, wherein the medicinal gas is NO, CO, hydrogen, oxygen, helium, xenon, H₂S, N₂O, argon, and any mixtures thereof.

9. The method according to claim 7, wherein the pressure of the gas in the introducing step is between 1.0 and 2.0 atm.

10. The method according to claim 7, wherein said PVA microbubbles are those made according to the method of claim 1.

11. The PVA microbubbles of claim 4, wherein the microbubbles are filled with a medicinal gas.

12. The PVA microbubbles according to claim 11, wherein the medicinal gas is NO, CO, hydrogen, oxygen, helium, xenon, H₂S, N₂O, argon, and any mixtures thereof.

13. The PVA microbubbles according to claim 11, wherein the gas is NO.

14. A method of using the PVA microbubbles of claim 12 as a medicament, comprising the step of administering the microbubbles to a patient in need of the medicinal gas.

15. A method of using the PVA microbubbles according to claim 12 as a diagnostic agent comprising the steps of administering the microbubbles to a patient and subsequently subjecting the patient to a diagnostic method.

16. A method of using the PVA microbubbles according to claim 12 as a contrast agent for ultrasound echography comprising the steps of administering the microbubbles to a patient and subsequently subjecting the patient to ultrasound imaging.

17. A method of using the PVA microbubbles according to claim 12 as an anticlotting agent comprising the step of administering the microbubbles to a patient in need of an anticlotting therapy.

18. A method of using the microbubbles according to claim 4 as a contrast agent for ultrasound echography comprising the steps of administering the microbubbles to a patient and subsequently subjecting the patient to ultrasound imaging.
